(19) United States
(12) Patent Application Publication
(10) Pub. No.: US 2006/0222608 A1
(43) Pub. Date: Oct. 5, 2006

(54) Edible tremella polysaccharide for skin care

(75) Inventors: Shu-Hui Yang, Kaohsiung (TW); Huey-Ing Liu, Taichung (TW); Shwu-Jene Tsai, Taichung (TW)

Correspondence Address: BACON & THOMAS, PLLC, 625 SLATERS LANE, FOURTH FLOOR, ALEXANDRIA, VA, 22314, US

(73) Assignee: AGRICULTURAL RESEARCH INSTITUTE, Taichung (TW)

(21) Appl. No.: 11/095,525

(22) Filed: Apr. 1, 2005

(51) Int. Cl. A61K 8/72; A61K 36/06; C12P 19/04

(52) U.S. Cl. 424/62; 424/195.15; 536/123

(57) ABSTRACT

It has been found that the polysaccharide isolated from a hot water extract of a Tremella mushroom without adding a chemical reagent has a novel effect of inhibiting melanin formation effects, so it can be used to lighten the spots on the skin when being applied to the skin. In addition, the Tremella polysaccharide of the present invention has excellent moisturizing effect.

### FIELD OF THE INVENTION

The present invention relates to a new use of the edible Tremella polysaccharide, and more particularly to a new use of the edible Tremella polysaccharide for skin care.

### BACKGROUND OF THE INVENTION

Mushrooms with distinctive fruiting bodies of sufficient size to be seen with the naked eye, include about 10,000 species of varying degrees of edibility. Approximately 100 species have been tested for cultivation and only seven to eight have been cultivated on an industrial scale. The world production of cultivated edible mushrooms in 1994 was estimated to be about five million tons and was valued at about ten billion US dollars. The most popular species of cultivated edible mushrooms include Agaricus bisporus (J. Lge) Imbach, A. bitorquis (Quel.) Sacc., Lentinus edodes (Berk) Sing., Pleurotus spp., Auricularia spp., Volvariella volvacea (Fr.) Sing., Flammulina velutipes (Fr.) Sing., Tremella fuciformis Berk., Hypsizygus marmoreus (Peck) Bigel., Pholita nameko (T. Ito) S. Ito et Imai, Grifola frondosa (Dicks.: Fr.) S. F. Gray, Hericium erinaceus (Bull.: Fr.) Pers., Dictyophora indusiata (Vent.: Pers.) Fischer, Stropharia nigosoannulata Far. apud Murr., Lepista nuda (Bull.: Fr.) Cooke, and Agrocybe aegerita (Brig) Sing.

The cultivation of fruiting bodies of mushrooms deals with living organisms, for example, the mushroom itself and other microorganisms which may either be harmful or beneficial. Therefore, the methods employed in mushroom cultivation require modifications depending upon the region being cultivated, substrates available, environmental conditions and species of microorganisms encountered.

The cultivation of mushrooms for fruiting bodies production is a long-term process needing from one to several months for the first fruiting bodies to appear. Moreover, it was found that processes for extraction of polysaccharides from fruiting bodies are not considered commercially feasible, since the physicochemical properties of the products resulting from these processes were not known or regulated (U.S. Pat. No. 4,051,314). Submerged culturing of polysaccharide producers allows obtaining the end product of constant composition in a short period under controlled conditions using ecologically pure culture medium of defined composition.

Tremella mushrooms belong to the so-called jelly mushrooms, which form gelatinous fruiting bodies. The jelly mushrooms are a set of species from different taxonomical groups of Phragmobasidiomycetes, which are able to survive long periods of drought by drying to a horny texture. When moisture is again available, they absorb water and become gelatinous. This characteristic of jelly mushrooms is due to the presence of specific water absorbing polysaccharides that compose 60-70% of the dry fruiting body. Unlike the β-1-3-glucans polysaccharides from other medicinal mushrooms, jelly mushroom polysaccharides consist of other sugars as well as glucose, and therefore belong to the class of heteropolysaccharides. A unique feature of Tremella mushrooms is that their pharmacologically active polysaccharides make up most of the structural fruiting body polysaccharides while in other medicinal mushrooms pharmacologically active polysaccharides make up only a small part of the biomass. For example, in shiutake mushrooms only 31 g of lentinan was extracted from 200 kg of fresh mushrooms, Mizuno, 1999, Int. J. Medicinal Mushrooms, 1:7-27.

The main pharmacologically active substance from Tremella is the polysaccharide glucuronoxylomannan, consisting of a linear backbone of 1,3-linked alpha-D-mannose with mainly xylose and glucuronic acid in side chains. The chemical structure of Tremella glucuronoxylomannan differs among various samples of even one species, and may be in some way connected with a type of polysaccharide-based method of identification. The general proportions of xylose:glucuronic acid:mannose are given in Tremella fuciformis as 1.0:2.77:4.9; 2:1:4 in T. aurantia, and 7:1:5 in T. mesenterica. Some additional saccharides can be identified in different samples of T. fuciformis, such as glucose and fucose, xylobiose and fructose.

Generally, Tremella glucuronoxylomannan has been found in cultivating different mushroom strains, that the polysaccharides extracted from the fruiting bodied and from mycelia in pure cultures are not essentially the same, although both may be pharmacologically active. A slight difference was observed in xylose: glucuronic acid: mannose proportions in Tremella fuciformis polysaccharide from fruiting bodies (1.0:2.77:4.9) and those from pure cultures of different haploid yeast-like budding strains (1:0.8-1.3:2.1-3.5).

Naturally growing or artificially fruiting bodies of Tremella mushrooms have been extensively used as a composition for dietary supplement (U.S. Pat. No. 6,383,799), stimulator of vascular endothelial cells (U.S. Pat. No. 5,616,325), anti-allergic drug (JP 1,228,480) or skin cosmetics (JP 61,289,011, JP 63,227,512, JP 3,099,005, JP 7,033,623, JP 7,126,149, JP 9,143,024).

In the present invention, a new effect on skin care of the Tremella polysaccharide is found.

### SUMMARY OF THE INVENTION

According to the present invention, it has been found that the polysaccharide isolated from a hot water extract of a Tremella mushroom without adding a chemical reagent has a novel effect of inhibiting melanin formation effects, so it can be used to lighten the spots on the skin when being applied to the skin. In addition, the Tremella polysaccharide of the present invention has excellent moisturizing effect.

The Tremella polysaccharide of the present invention is a glucuronoxylomannan, an acid heteropolysaccharide, and has a linear backbone of α-(1→3)-D-mannan, substituted with β-D-xylose, β-D-glucuronic acid and β(1→2) D-xylobiose at C2 position of mannose residue and a molecular weight of 0.5-6.0×10⁶ dalton.

The Tremella polysaccharide of the present invention is a colorless, transparent, odorless, tasteless, and viscous material, and can form a thin film on the skin.

The above objects and advantages of the present invention will become more readily apparent to those ordinarily skilled in the art after reviewing the following detailed description.

### DETAILED DESCRIPTION OF THE PREFERRED EMBODIMENT

The present invention provides a new use of the Tremella polysaccharide for skin care. The polysaccharide is isolated from the Tremella mushroom including but not limited to Tremella fuciformis Berk, Tremella mesenterica, Tremella aurantia, and Tremella encepuala.

In order to obtain a more natural extract of Tremella mushroom, a physical method is used to extract the active polysaccharide in the present invention without adding a chemical agent. The method is described as follows. First, a raw material of the fruiting bodies of Tremella fuciformis, Berk is rinsed with water. Subsequently, a suitable amount of water is added to the raw material, and then heated to 80-175° C. to extract the polysaccharide in hot water. After the hot water extraction for about 0.5-4 hours, it is centrifuged at 3000-5000 rpm for about 2-5 minutes to obtain a polysaccharide solution, which is colorless, transparent, odorless, tasteless, and viscous. Besides, the residues can be resuspended in the water and then heated and centrifuged as the above steps to obtain more polysaccharide extracts.

The extracted polysaccharide is glucuronoxylomannan, an acid heteropolysaccharide, having a linear backbone of α-(1→3)-D-mannan, substituted with β-D-xylose, β-D-glucuronic acid and β-(1→2) D-xylobiose at C2 position of mannose residue, and its molecular weight is 0.5-6.0×10⁶ dalton. This high-molecular substance shows good viscosity at temperature of 1-100° C. and can be stored for a long period of time. The extracted polysaccharide is mild to our skin; when it is applied to the skin, it makes the skin soft and tender. The extracted polysaccharide can form a thin film on the skin, which makes the skin bright and shining. In addition, it significantly reduces water evaporation and increases moisture retention on the skin, so it can be used as a major ingredient of a moisturizing cosmetic, or added to other cosmetics, such as lotions, creams, masks, and essences, to increase their moisturizing effects. More particular, the extracted polysaccharide of the present invention remarkably suppresses melanogenesis without manifesting cytotoxicity by way of inhibiting melanin formation. Therefore, the Tremella polysaccharide of the present invention can be used as a spot eraser to lighten the spots on the face and improve the brightness of the skin. Furthermore, it is found that the Tremella polysaccharide of the present invention has potentials of inhibiting the secretion of facial oil and soothing the symptom of seborrheic dermatitis.

It is an advantage of the present invention that the Tremella polysaccharide is extracted by a physical method without adding a chemical reagent, so the derived product is quite natural and the production yield is high. Moreover, the extracted polysaccharide solution can be directly applied to the skin without any post processing. The following examples show the effects on skin care of the Tremella polysaccharide according to the present invention.

### EXAMPLE 1

### Effect of Retaining Skin Moisture

The test sample is the extracted polysaccharide solution of the present invention, and the voluntary testers are females aged 20-50 and with no medical history of allergy. The voluntary testers are divided into two groups, the group of normal skin and the group of dry skin. The skin locations to be tested, which are the inner side of the arm, are applied with no cosmetic or drug during 48 hours before test, and are washed with low stimulative soap before test and then wiped. After that, the testers take a rest for 30 minutes at constant temperature of 20±1° C. and relative humidity of 45-50%. Five test positions are labeled, and each has a diameter of 3 cm. Then, 100 μl of the test sample is evenly applied to the labeled positions (using distilled water as the blank experiment). The water content in the stratum corneum of the skin is analyzed by Skin Analyzer SHP 88 every 30 minutes, and the water evaporation of the skin is analyzed by Tewameter TM210 every 60 minutes for total 3 hours. The results are shown in the following Tables 1-3.

TABLE 1
Water content in the stratum corneum of the skin

| | Time | 30 min | 60 min | 90 min | 120 min | 150 min | 180 min |
|---|---|---|---|---|---|---|---|
| Normal | Distilled | 09.0 ± 0.0 | 09.0 ± 0.3 | 08.7 ± 0.2 | 08.5 ± 0.2 | 08.0 ± 0.3 | 08.0 ± 0.3 |
| Skin | Waster | | | | | | |
| | Tremella | 12.2 ± 0.9 | 12.5 ± 0.8 | 12.0 ± 0.7 | 12.8 ± 0.8 | 11.3 ± 1.1 | 12.5 ± 0.6 |
| | Polysaccharide | | | | | | |
| Dry | Distilled | 07.8 ± 0.2 | 07.3 ± 0.2 | 07.3 ± 0.2 | 07.3 ± 0.3 | 07.2 ± 0.2 | 07.3 ± 0.2 |
| Skin | Waster | | | | | | |
| | Tremella | 09.8 ± 0.7 | 10.5 ± 0.8 | 09.5 ± 0.6 | 08.8 ± 0.5 | 09.5 ± 1.1 | 09.5 ± 0.8 |
| | Polysaccharide | | | | | | |

Mean ± S.E(n = 6)
unit: a.u.

TABLE 2
Increasing ratio of water content in the stratum corneum of the normal
skin

| | Time | | | | | |
|---|---|---|---|---|---|---|
| | 30 min | 60 min | 90 min | 120 min | 150 min | 180 min |
| Tremella | 35.6% | 38.9% | 37.9% | 50.6% | 41.3% | 56.3% |
| Polysaccharide | | | | | | |

TABLE 3
Increasing ratio of water content in the stratum corneum of the dry skin

| | Time | | | | | |
|---|---|---|---|---|---|---|
| | 30 min | 60 min | 90 min | 120 min | 150 min | 180 min |
| Tremella | 25.6% | 43.8% | 30.1% | 20.5% | 31.9% | 30.1% |
| Polysaccharide | | | | | | |

When applying the Tremella polysaccharide of the present invention to the normal skin, the water content in the stratum corneum of the skin stably keeps at about 12 a.u. without significant reduction from the beginning of the test to 180 minutes thereafter, as shown in Table 1; while in the water blank experiment, the water content in the stratum corneum of the skin is significantly reduced. The water content in the stratum corneum of the dry skin is apparently lower than that of the normal skin, but the water content in the stratum corneum of the skin still stably keeps at about 9.5 a.u. from the beginning of the test to 180 minutes thereafter; while in the water blank experiment, the water content in the stratum corneum of the skin is reduced. Therefore, the Tremella polysaccharide of the present invention shows good and stable water retaining ability and has excellent moisturizing effect.

Comparing the normal skins applied with or without the Tremella polysaccharide, the increasing ratio of the water content in the stratum corneum of the normal skin when applied with the Tremella polysaccharide in respect to that applied with water is about 35-56%, as shown in Table 2, and as the time passing to 180 minutes, the increasing ratio tends to be increased. On the other hand, the increasing ratio of the water content in the stratum corneum of the dry skin when applied with the Tremella polysaccharide in respect to that applied with water is about 20-44%, as shown in Table 3.

### EXAMPLE 2

### Effect of Inhibiting Melanin Formation

The test sample is the extracted polysaccharide solution of the present invention, and the effect of inhibiting the melanin formation is determined via dopachrome (dopa chrome), which is an intermediate product in the biosynthesis process from tyrosine to melanin.

First, four sample vials are prepared and labeled with At, Al, Ab and A0, respectively. 0.9 ml buffer solution is added into the four vials respectively, and then 1 ml tyrosine solution is added into the four vials respectively. After that, 1 ml test sample is added into At and A1 vials respectively, and 1 ml distilled-deionized water is added into Ab and A0 vials respectively. The four vials are put into the 37° C. water bath for 10 minutes. Subsequently, 0.1 ml buffer solution is added into A1 and A0 vials respectively, and 0.1 ml tyrosinase solution is added into At and Ab vials respectively. Then the four vials are put into the 37° C. water bath for 25 minutes. Finally, the absorption at the wavelength(λ) of 475 nm of each vial is determined by a UV-visible light spectrum meter.

The inhibition ratio of the melanin formation is calculated according to the following equation:

$$Inhibition ratio ( \% ) = \frac{( Ab - A 0 ) - ( At - A 1 )}{( Ab - A 0 )} \times 100 \%$$

wherein At represents the absorption of dopachrome in the melanin biosynthesis when added with the test sample, A1 represents the adsorption of the test sample, Ab represents the adsorption of dopachrome in the melanin biosynthesis, and A0 represents the adsorption of the solvent.

It is resulted that the Tremella polysaccharide of the present invention has an effect of inhibiting the melanin formation with an inhibition ratio of 59.7%. To be compared with other ingredients in the cosmetics which are claimed to have the spot lightening effect, such as Arbutin, Kojic acid and Vitamine C, they are tested by the same method, and the inhibition ratios thereof are respectively 97.9%, 99.2% and 99.1%. Therefore, the Tremella polysaccharide of the present invention has a much better effect of inhibiting the melanin formation, and thus it can be used to lighten the spots on the skin and improve the skin luster. Therefore, it can be applied to a functional cosmetic, which is a new use of the edible Tremella polysaccharide.

In conclusion, the Tremella polysaccharide of the present invention is extracted via hot water without adding any chemical reagent, so it is more natural and can be applied to the skin directly. The Tremella polysaccharide of the present invention has excellent moisturizing effect, and more particular, it can inhibit the melanin formation, so it can be used as a spot eraser to lighten the spots on the skin and improve the brightness of the skin. In addition, the Tremella polysaccharide shows good viscosity and can be stored for a long period of time. Therefore, the Tremella polysaccharide is a new cosmetic material which has good effects of retaining skin moisture and inhibiting melanin formation.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A polysaccharide isolated from a hot water extract of a Tremella mushroom without adding a chemical reagent, having effects of retaining skin moisture and inhibiting melanin formation.

2. The polysaccharide according to claim 1 being an acid heteropolysaccharide.

3. The polysaccharide according to claim 2 wherein said acid heteropolysaccharide is a glucuronoxylomannan.

4. The polysaccharide according to claim 1 wherein the structure of said polysaccharide has a linear backbone of α-(1→3)-D-mannan, substituted with β-D-xylose, β-D-glucuronic acid and β-(1→2) D-xylobiose at C2 position of mannose residue.

5. The polysaccharide according to claim 1 wherein said polysaccharide has a molecular weight of 0.5-6.0×10⁶ dalton.

6. The polysaccharide according to claim 1 wherein said polysaccharide is a colorless, transparent, odorless, tasteless, and viscous material.

7. The polysaccharide according to claim 1 wherein said polysaccharide forms a thin film on the skin.

8. A method of lightening spots on the skin, comprising applying a polysaccharide to the skin, wherein said polysaccharide is isolated from a hot water extract of Tremella mushroom without adding a chemical reagent.

9. The polysaccharide according to claim 8 wherein said polysaccharide is an acid heteropolysaccharide.

10. The method according to claim 9 wherein said acid heteropolysaccharide is a glucuronoxylomannan.

11. The method according to claim 8 wherein the structure of said polysaccharide has a linear backbone of α(1→3)-D-mannan, substituted with β-D-xylose, β-D-glucuronic acid and β-(1→2) D-xylobiose at C2 position of mannose residue.

12. The method according to claim 8 wherein said polysaccharide has a molecular weight of 0.5-6.0×10⁶ dalton.

13. The method according to claim 8 wherein said polysaccharide is a colorless, transparent, odorless, tasteless, and viscous material.

14. The method according to claim 1 wherein said polysaccharide forms a thin film on the skin.

15. A method of obtaining a polysaccharide having effect of inhibiting melanin formation comprising isolating said polysaccharide from a hot water extract of Tremella mushroom without adding a chemical reagent.
